(19) United States
(12) Patent Application Publication
(10) Pub. No.: US 2018/0369507 A1
(43) Pub. Date: Dec. 27, 2018

(54) MEDICAL NEBULIZER WITH IMPROVED STRUCTURE

(71) Applicant: NINGBO BESMED MEDICAL EQUIPMENT CORP., Zhejiang (CN)

(72) Inventors: Tao-Tsun HSIUNG, New Taipei City (TW); Xu-Xiang WANG, New Taipei City (TW)

(21) Appl. No.: 15/667,061

(22) Filed: Aug. 2, 2017

(30) Foreign Application Priority Data
Jun. 23, 2017 (TW) 10 6121339
Jun. 23, 2017 (TW) 10 6209301

(51) Int. Cl. A61M 11/00; A61M 11/06; A61M 11/02; B05B 7/00

(52) U.S. Cl. CPC A61M 11/002; B05B 7/0012; A61M 11/02; A61M 11/06

(57) ABSTRACT

A medical nebulizer with improved structure, the medical nebulizer comprising a first housing and a second housing, the first housing integrally formed a first nozzle, the first nozzle has a first nozzle hole, the second housing integrally formed a second nozzle and a conical cover, the second nozzle has a second nozzle hole, the inside of the conical cover has a protrusion and an annular inclined surface, the protrusion formed a impacting surface to produce aerosol; wherein the second housing is separable assembled to the first housing, the second nozzle is located on the outside of the first nozzle, a fluid passage is formed between the first nozzle and the second nozzle, the first nozzle hole is located on the inside of the second nozzle hole, the impacting surface is located above the second nozzle hole.

### BACKGROUND OF THE INVENTION

### 1. Field of the Invention

The invention relates to a medical nebulizer and more particularly to a medical nebulizer having capability for reducing the remaining amount of liquid medication, the medical nebulizer has an improved structure which simplifies the medical nebulizer structure into two integrally formed housings, therefore the medical nebulizer can ensure the accuracy of product assembly and atomization effect, the medical nebulizer has a conical cover to recycle larger aerosol particles, and the larger aerosol particles can be atomized again.

### 2. Description of Related Art

A first prior-art is disclosed in U.S. Pat. No. 4,512,341 (its patent family has United Kingdom Patent No. GB2132508B), the first prior-art disclosed a nebulizer, the feed of the nebulizer to a spray nozzle is through a narrow space between the flat bottom of the liquid reservoir and a flange fixed to the bottom of the spray nozzle, wherein the space is narrow enough to draw liquid toward the spray nozzle by capillary action. When the liquid passed through a rounded diffuser and an inverted conical portion at high speed, the liquid will impact to a baffle plate to produce an aerosol, larger droplets are coalesced by the baffle plate, therefore the larger droplets can fall down to the liquid reservoir. The rounded diffuser may cause larger droplets to spray on a main body wall of the nebulizer, therefore the larger droplets will partially remain on the main body wall. Moreover, according to the description of the first prior-art, the flange located on the bottom of the spray nozzle almost extends to the wall surrounding the liquid reservoir, and when the water level of the liquid approaches the flange, the larger droplets will fall down to the flange, and the larger droplets will remain on the upside of the flange. Thus, the first prior-art still requires improvement to further reduce the remaining amount of liquid medication.

A second prior-art is disclosed in U.S. Pat. No. 5,738,086 (its patent family has Australia Publication No. AU1181595A, Canadian Publication No. CA2157581A1, EPO Publication No. EP0691862A1, Japan Publication No. JPH08508671A, New Zealand Publication No. NZ276953A, U.S. Pat. No. 5,579,757, PCT Publication No. WO9520989A1), the second prior-art disclosed a nebulizer, the nebulizer has a liquid spray nozzle and a nebulizer diffuser, the liquid spray nozzle is a separate element. When the nebulizer is assembled, there is a distance between a fluid orifice of the liquid spray nozzle and the nebulizer diffuser, the distance may exist a difference on the combination, that may affect the atomization effect. Moreover, according to the description of the second prior-art, an annular flange extends inwardly around the top of the second baffle, and a pair of diffuser legs having longitudinal spaces extend downward from the inwardly extending annular flange to support the nebulizer diffuser. The nebulizer diffuser is exposed to the second baffle, the nebulizer diffuser may cause larger droplets to spray on the inner wall of the nebulizer, therefore the larger droplets will partially remain on the inner wall. Thus, the second prior-art still requires improvement to further reduce the remaining amount of liquid medication.

### SUMMARY OF THE INVENTION

It is therefore a first object of the invention to provide a medical nebulizer with improved structure comprising a first housing and a second housing, the first housing integrally formed a first nozzle, the first nozzle has a first nozzle hole, the second housing integrally formed a second nozzle and a conical cover, the second nozzle has a second nozzle hole, the inside of the conical cover has a protrusion and an annular inclined surface, the protrusion formed a impacting surface to produce aerosol; wherein the second housing is separable assembled to the first housing, the second nozzle is located on the outside of the first nozzle, a fluid passage is formed between the first nozzle and the second nozzle, the first nozzle hole is located on the inside of the second nozzle hole, the impacting surface is located above the second nozzle hole.

It is therefore a second object of the invention to provide a medical nebulizer with improved structure comprising a first housing and a second housing, the first housing integrally formed a first nozzle, the first nozzle has a first nozzle hole, the second housing integrally formed a second nozzle and a conical cover, the second nozzle has a second nozzle hole, the inside of the conical cover has an annular inclined surface and a impacting surface for producing aerosol; wherein the second housing is separable assembled to the first housing, the second nozzle is located on the outside of the first nozzle, a fluid passage is formed between the first nozzle and the second nozzle, the first nozzle hole is located on the inside of the second nozzle hole, the impacting surface is located above the second nozzle hole.

First advantages of the invention include a second nozzle and a conical cover are integrally formed on the second housing, a distance existed between a impacting surface of producing aerosol and a second nozzle hole, the distance is determined by the mold, therefore the distance will not exist a difference on the combination. Thus the medical nebulizer can ensure the accuracy of product assembly and atomization effect.

Second advantages of the invention include the impacting surface of producing aerosol is located on the inside of the conical cover, most of larger droplets will be sprayed to an annular inclined surface of the conical cover, and therefore can reduce the droplets to attach to the inner wall of the medical nebulizer.

Third advantages of the invention include the conical cover can form a suitable space of aerosol escaping by the annular inclined surface and the second nozzle, the annular inclined surface is helpful to the collection of larger droplets and the dropping of larger droplets. Thus, the invention is optimized between the escaping amount of aerosols and the collection amount of larger droplets.

The above and other objects, features and advantages of the invention will become apparent from the following detailed description taken with the accompanying drawings.

### BRIEF DESCRIPTION OF THE DRAWINGS

FIG. 1 is a perspective view showing a first preferred embodiment of the invention;

FIG. 2 is an exploded view showing the first preferred embodiment of the invention;

FIG. 3 is a cross-sectional perspective view along a line A-A of FIG. 1 showing the first preferred embodiment of the invention;

FIG. 4 is a cross-sectional view along a line B-B of FIG. 1 showing the first preferred embodiment of the invention;

FIG. 5 is an operation diagram illustrating the first preferred embodiment of the invention;

FIG. 6 is a cross-sectional view along a line C-C of FIG. 1 showing the first preferred embodiment of the invention;

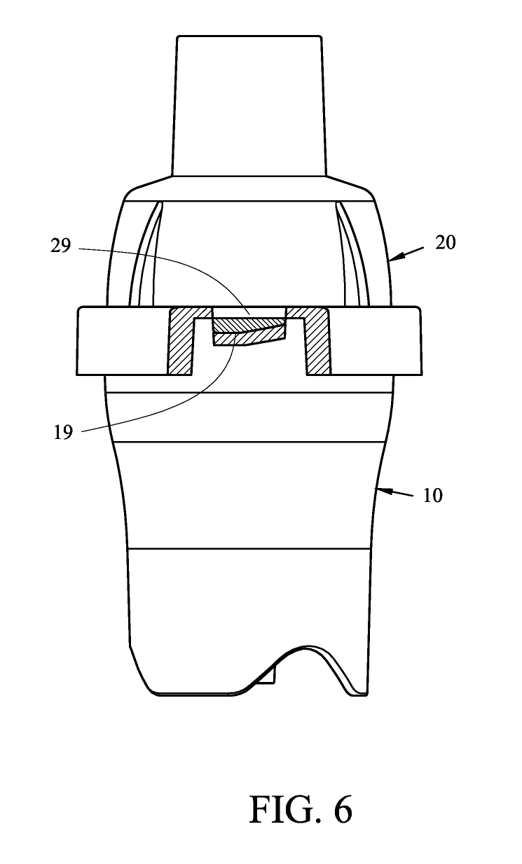

FIG. 7 is another perspective view showing a second housing of the first preferred embodiment of the invention;

FIG. 8 is a cross-sectional perspective view along a line D-D of FIG. 7 showing the first preferred embodiment of the invention;

FIG. 9 is another perspective view showing a first housing of the invention;

FIG. 10 is a cross-sectional perspective view along a line E-E of FIG. 9 showing the first preferred embodiment of the invention.

FIG. 11 is a perspective view showing a second embodiment of the invention;

FIG. 12 is an exploded view showing the second embodiment of the invention;

FIG. 13 is a cross-sectional perspective view along a line A-A of FIG. 11 showing the second embodiment of the invention;

FIG. 14 is a cross-sectional view along a line B-B of FIG. 11 showing the second embodiment of the invention;

FIG. 15 is an operation diagram illustrating the second embodiment of the invention;

FIG. 16 is a cross-sectional view along a line C-C of FIG. 11 showing the second embodiment of the invention;

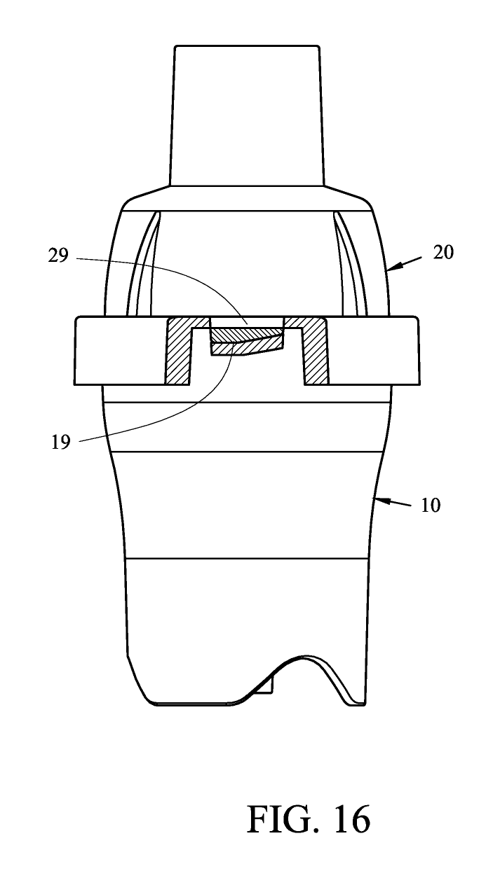

FIG. 17 is another perspective view showing a second housing of the second embodiment of the invention;

FIG. 18 is a cross-sectional perspective view along a line D-D of FIG. 17 showing the second embodiment of the invention;

FIG. 19 is another perspective view showing a first housing of the second embodiment of the invention;

FIG. 20 is a cross-sectional perspective view along a line E-E of FIG. 19 showing the second embodiment of the invention.

### DETAILED DESCRIPTION OF THE INVENTION

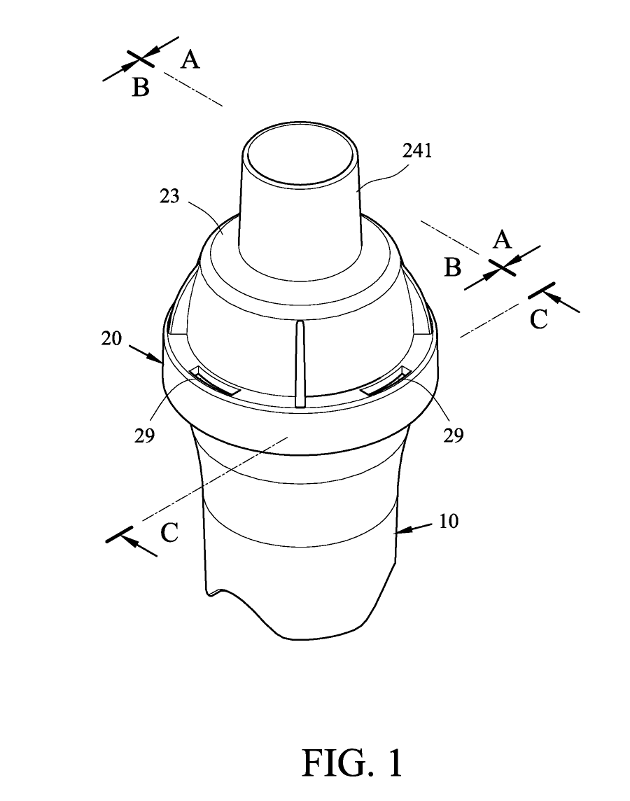

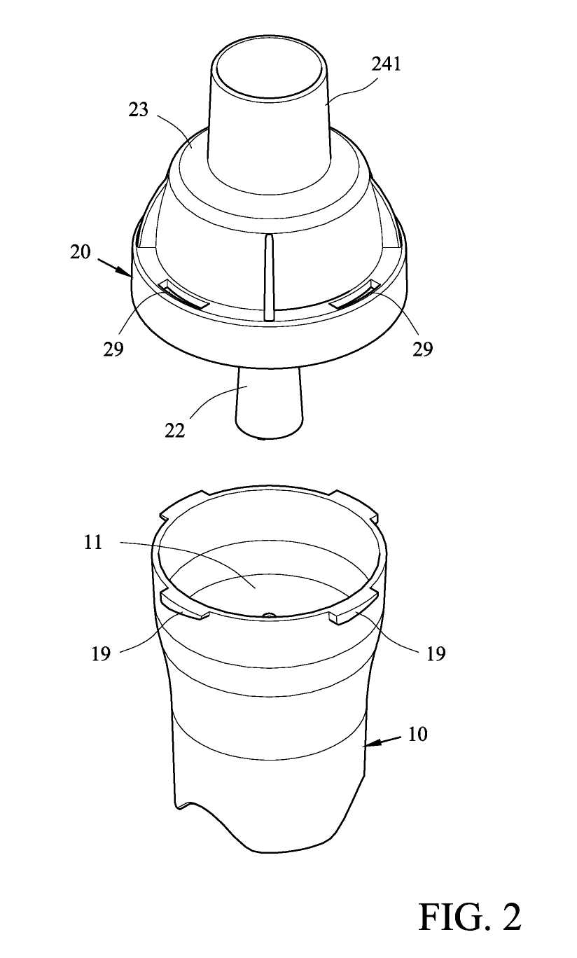

Referring to FIGS. 1 to 4, a medical nebulizer in accordance with a first preferred embodiment of the invention comprises a first housing 10 and a second housing 20, the first housing 10 has a first containing space 11, the first containing space 11 is provided with a first nozzle 12, the first nozzle 12 has a first nozzle hole 120, a bottom portion 13 of the first housing 10 outwardly extends a gas inlet tube 14, the gas inlet tube 14 is fluid communicated with the first nozzle 12; the second housing 20 is provided with a second nozzle 22, the second nozzle 22 has a second nozzle hole 220, the top portion 23 of the second housing 20 has a aerosol outlet tube 24, a conical cover 25 is connected with the aerosol outlet tube 24, the second nozzle 22 is connected with the conical cover 25, the inside of the conical cover 25 has a protrusion 251 and an annular inclined surface 252, the protrusion 251 formed a impacting surface 253 to produce aerosol; wherein the second housing 20 is separable assembled to the first housing 10, the second nozzle 22 is located on the outside of the first nozzle 12, a fluid passage 26 is formed between the first nozzle 12 and the second nozzle 22, the first nozzle hole 120 is located on the inside of the second nozzle hole 220, the impacting surface 253 is located above the second nozzle hole 220, a first pitch D1 is formed between the impacting surface 253 and the second nozzle hole 220, a second pitch D2 is formed between the annular inclined surface 252 and the top end 222 of the second nozzle 22, the position of the second nozzle hole 220 is higher than the bottom edge 254 of the annular inclined surface 252.

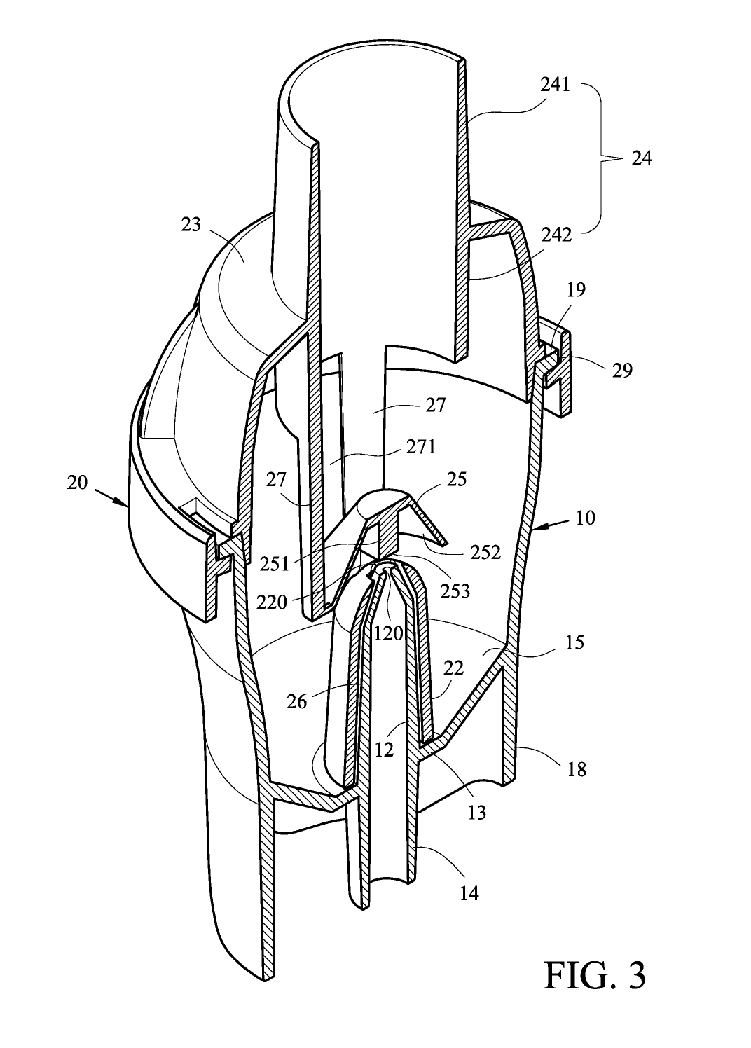

Referring to FIGS. 3 to 5, examples of the operation manner of the invention will be illustrated below. When the second housing 20 is separated from the first housing 10, a liquid 30 having medication can be poured into the first containing space 11 of the first housing 10. When the first housing 10 is assembled to the second housing 20, the gas inlet tube 14 can connect with a tube 40, and the tube 40 can connect with a gas supply source (not shown) to input gas, the gas forms a gas flow 41 in the second nozzle 22. When the gas flow 41 passes through the first nozzle hole 120, the gas pressure will be changed in the vicinity of the first nozzle hole 120, the liquid 30 can be drawn to the fluid passage 26, the gas flow 41 can drive the liquid 30 to pass through the second nozzle hole 220 to form a jet flow (not shown), the jet flow will produce a plurality of aerosols 31 and larger droplets 32 after the jet flow impacted the impacting surface 253.

Examples of the manufacture manner of the first housing 10 and the second housing 20 will be illustrated below, the first nozzle 12 and the gas inlet tube 14 are integrally formed on the first housing 10, the second nozzle 22 and the conical cover 25 are integrally formed on the second housing 20; wherein the first pitch D1 formed between the impacting surface 253 and the second nozzle hole 220 is determined by the mold, therefore the first pitch D1 will not exist a difference on the combination. Thus the invention can ensure the accuracy of product assembly and atomization effect.

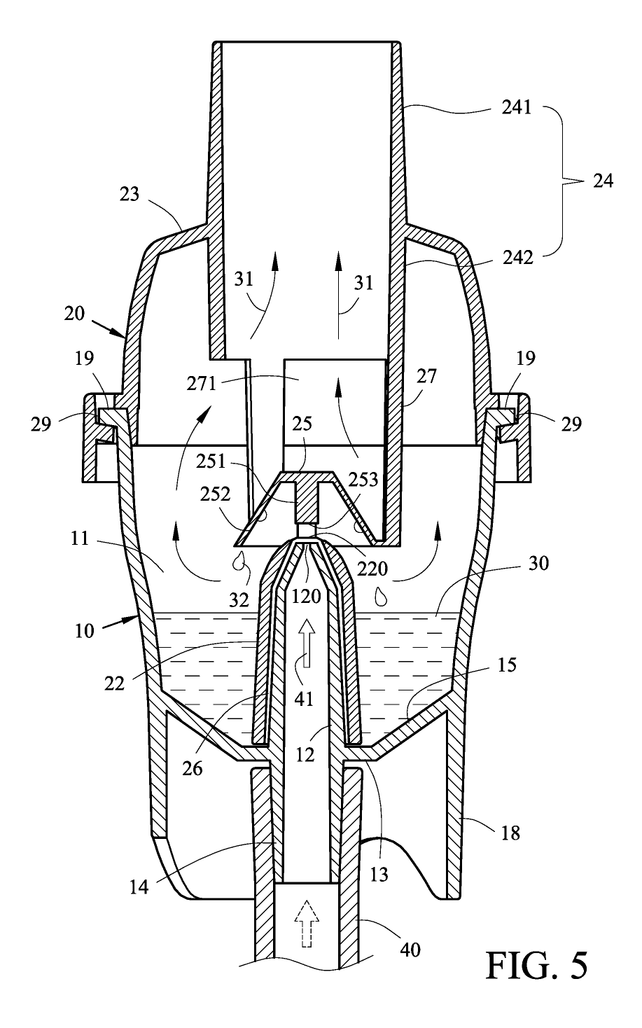

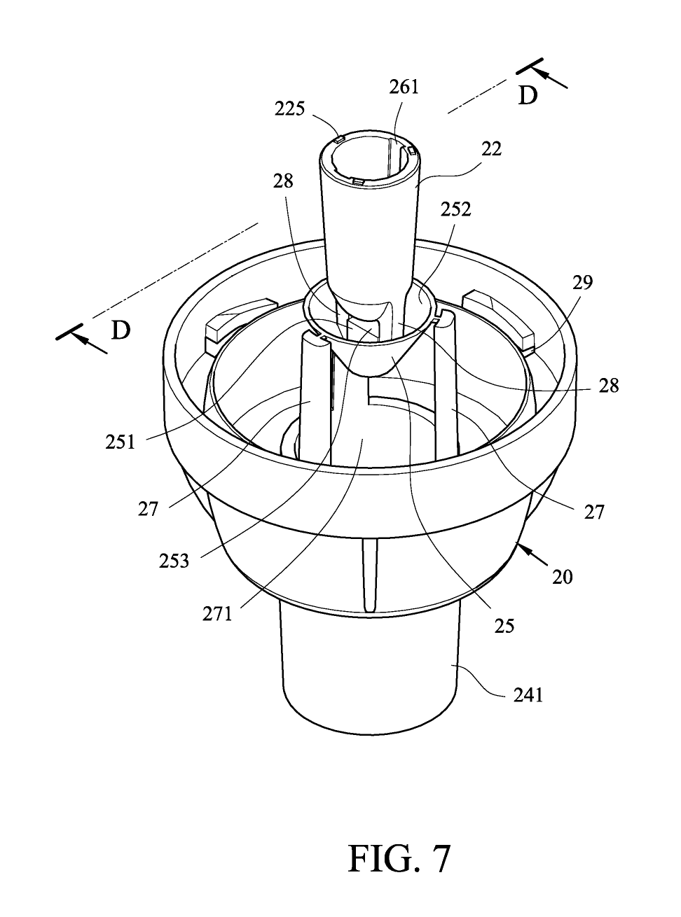

Examples of the connecting manner of the aerosol outlet tube 24 and the conical cover 25 will be illustrated below, the aerosol outlet tube 24 is connected to the conical cover 25 by a plurality of first connecting ribs 27, each pair of the adjacent connecting ribs 27 can form an aerosol window 271, the height of the conical cover 25 is in the range of 3 mm to 8 mm; wherein the aerosols 31 can enter the aerosol outlet tube 24 from the aerosol window 271, and the aerosols 31 can be outputted from the aerosol outlet tube 24 (as shown in FIG. 5 and FIG. 7).

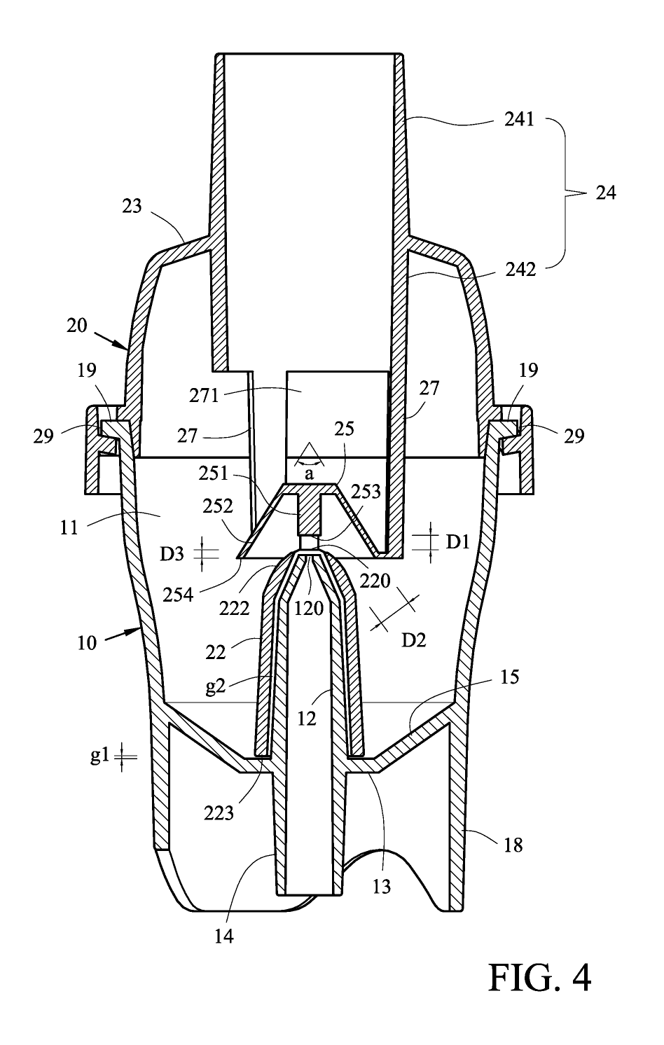

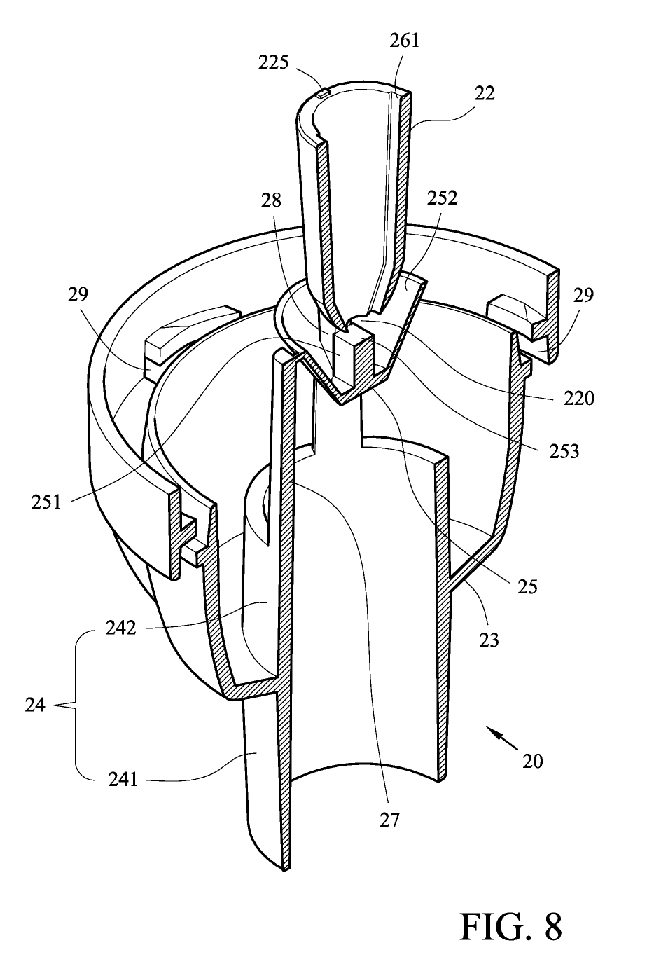

Examples of the connecting manner of the second nozzle 22 and the conical cover 25 will be illustrated below, the conical cover 25 is connected to the second nozzle 22 by a plurality of second connecting ribs 28 (as shown in FIG. 7 to FIG. 8). Moreover, the first pitch D1 formed between the impacting surface 253 and the second nozzle hole 220 is in the range of 1 mm to 3 mm, the second pitch D2 formed between the annular inclined surface 252 and the top end 222 of the second nozzle 22 is in the range of 1.5 mm to 5 mm, the impacting surface 253 is located on the inside of the conical cover 25, most of larger droplets 32 will be sprayed to the annular inclined surface 252 of the conical cover 25, and therefore can reduce the droplets 32 to attach to the inner wall of the medical nebulizer. Thus, the invention can further reduce the remaining amount of the liquid 30 (as shown in FIG. 4 to FIG. 5).

Examples of the positional relationship of the second nozzle hole 220 and the annular inclined surface 252 will be illustrated below, a third pitch D3 is formed between the second nozzle hole 220 and the bottom edge 254 of the annular inclined surface 252, the third pitch D3 is in the range of 0.1 mm to 3 mm, the annular inclined surface 252 of the conical cover 25 forms an angle a, the angle a is in the range of 40 degrees to 70 degrees; the conical cover 25 can form a suitable space of aerosol escaping by the annular inclined surface 252 and the second nozzle 22, the annular inclined surface 252 is helpful to the collection of larger droplets 32 and the dropping of larger droplets 32. Thus, the invention is optimized between the escaping amount of aerosols 31 and the collection amount of larger droplets 32 (as shown in FIG. 3 to FIG. 5).

Examples of a first practice manner of the fluid passage 26 will be illustrated below, a first gap g1 is formed between an end edge 223 of the second nozzle 22 and the bottom portion 13 of the first housing 10, the fluid passage 26 can be formed by a second gap g2 which located between the first nozzle 12 and the second nozzle 22 (as shown in FIG. 3 to FIG. 5).

Examples of a second practice manner of the fluid passage 26 will be illustrated below, a first gap g1 is formed between an end edge 223 of the second nozzle 22 and the bottom portion 13 of the first housing 10, the second nozzle 22 is provided with a plurality of inner grooves 261 to form the fluid passage 26 (as shown in FIG. 4, FIG. 5, FIG. 7 and FIG. 8).

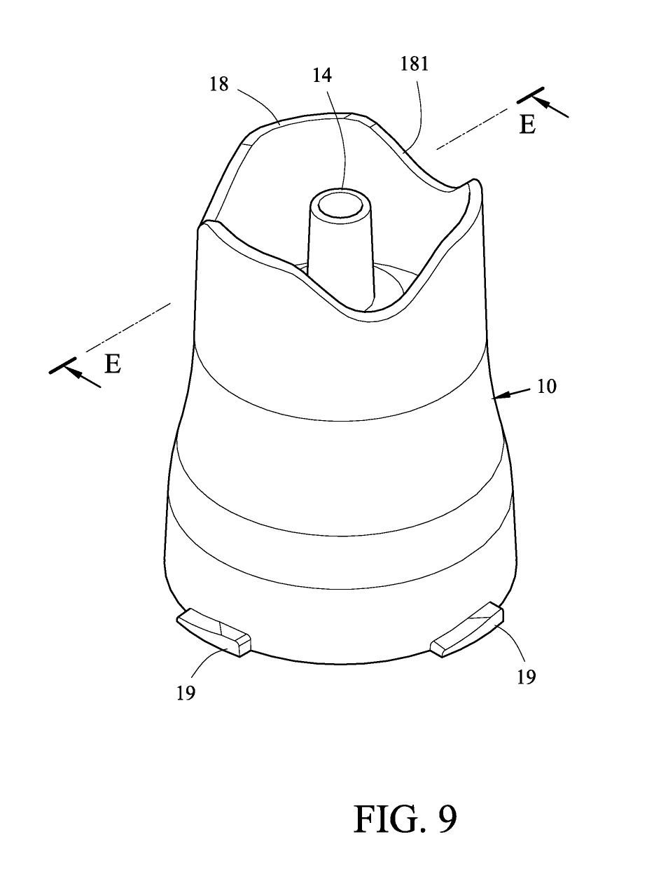

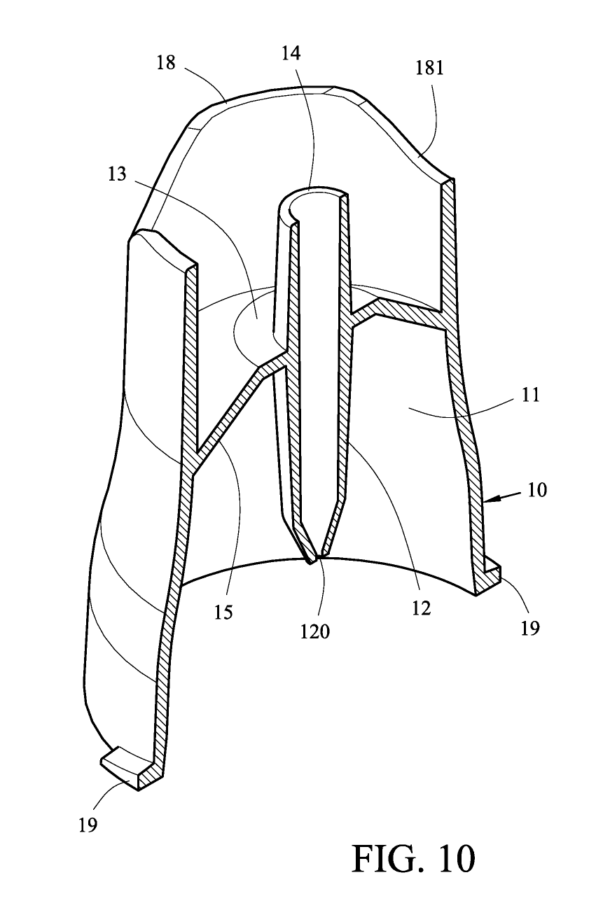

Examples of the structural change of the first housing 10 and the second housing 20 will be illustrated below, the bottom portion 13 of the first housing 10 has an inclined extension surface 15 to reduce the remaining amount of the liquid 30 (as shown in FIG. 9 to FIG. 10). Moreover, the end edge 223 of the second nozzle 22 has a plurality of blocks 225 to ensure a smallest distance of the first gap g1 (as shown in FIG. 4, FIG. 7 and FIG. 8). Furthermore, the first housing 10 further has an annular stand 18, the length of the annular stand 18 is larger than the length of the gas inlet tube 14, and the annular stand 18 has a plurality of notches 181 (as shown in FIG. 9 to and FIG. 10).

Examples of the structural change of the second housing 20 will be illustrated below, the aerosol outlet tube 24 has a first outlet tube 241 and a second outlet tube 242, the first outlet tube 241 is located on outside of the top portion 23 of the second housing 20, the second outlet tube 242 is located on inside of the top portion 23 of the second housing 20; when the medical nebulizer of the invention is tilted, the second outlet tube 242 can prevent the liquid 30 flow from the second housing 20 (as shown in FIG. 3 to and FIG. 8).

Examples of the combination manner of the first housing 10 and the second housing 20 will be illustrated below, the first housing 10 has a plurality of positioning blocks 19, the second housing 20 has a plurality of positioning slots 29; when the first housing 10 and the second housing 20 relatively rotate to each other, each of the positioning blocks 19 can enter into each of the positioning slots 29, or each of the positioning blocks 19 can escape from each of the positioning slots 29 (as shown in FIG. 3 to and FIG. 10).

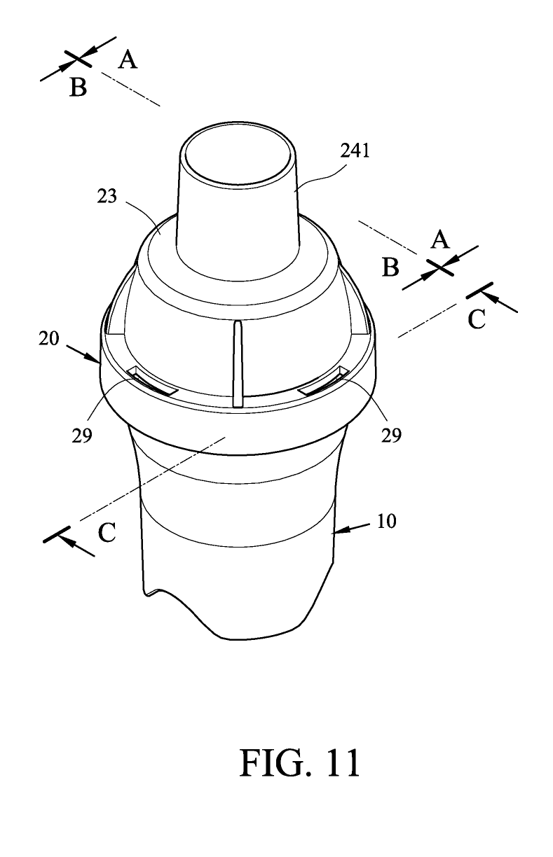

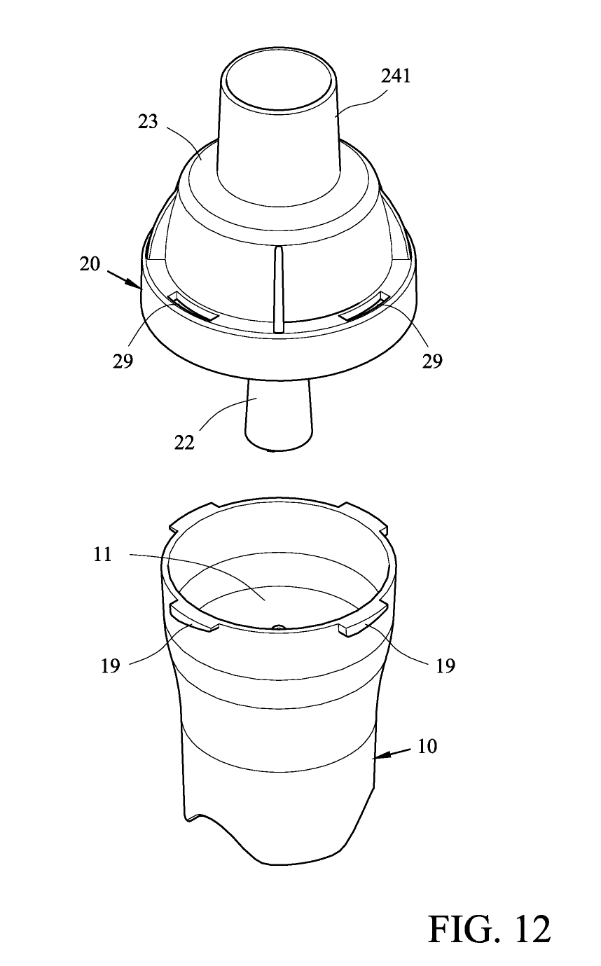

Referring to FIGS. 11 to 14, a medical nebulizer in accordance with a second embodiment of the invention comprises a first housing 10 and a second housing 20, the first housing 10 has a first containing space 11, the first containing space 11 is provided with a first nozzle 12, the first nozzle 12 has a first nozzle hole 120, a bottom portion 13 of the first housing 10 outwardly extends a gas inlet tube 14, the gas inlet tube 14 is fluid communicated with the first nozzle 12; the second housing 20 is provided with a second nozzle 22, the second nozzle 22 has a second nozzle hole 220, the top portion 23 of the second housing 20 has a aerosol outlet tube 24, a conical cover 25 is connected with the aerosol outlet tube 24, the second nozzle 22 is connected with the conical cover 25, the inside of the conical cover 25 has an annular inclined surface 252 and a impacting surface 253 for producing aerosol; wherein the second housing 20 is separable assembled to the first housing 10, the second nozzle 22 is located on the outside of the first nozzle 12, a fluid passage 26 is formed between the first nozzle 12 and the second nozzle 22, the first nozzle hole 120 is located on the inside of the second nozzle hole 220, the impacting surface 253 is located above the second nozzle hole 220, a first pitch D1 is formed between the impacting surface 253 and the second nozzle hole 220, a second pitch D2 is formed between the annular inclined surface 252 and the top end 222 of the second nozzle 22, the position of the second nozzle hole 220 is higher than the bottom edge 254 of the annular inclined surface 252.

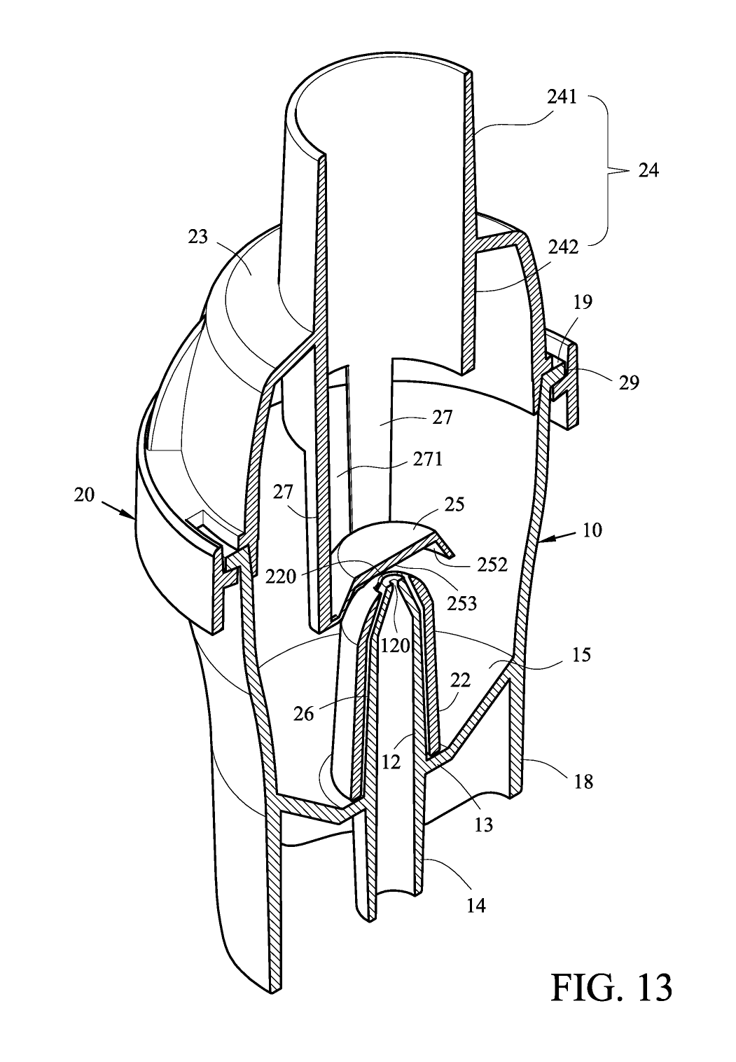

Referring to FIGS. 13 to 15, examples of the operation manner of the invention will be illustrated below. When the second housing 20 is separated from the first housing 10, a liquid 30 having medication can be poured into the first containing space 11 of the first housing 10. When the first housing 10 is assembled to the second housing 20, the gas inlet tube 14 can connect with a tube 40, and the tube 40 can connect with a gas supply source (not shown) to input gas, the gas forms a gas flow 41 in the second nozzle 22. When the gas flow 41 passes through the first nozzle hole 120, the gas pressure will be changed in the vicinity of the first nozzle hole 120, the liquid 30 can be drawn to the fluid passage 26, the gas flow 41 can drive the liquid 30 to pass through the second nozzle hole 220 to form a jet flow (not shown), the jet flow will produce a plurality of aerosols 31 and larger droplets 32 after the jet flow impacted the impacting surface 253.

Examples of the manufacture manner of the first housing 10 and the second housing 20 will be illustrated below, the first nozzle 12 and the gas inlet tube 14 are integrally formed on the first housing 10, the second nozzle 22 and the conical cover 25 are integrally formed on the second housing 20; wherein the first pitch D1 formed between the impacting surface 253 and the second nozzle hole 220 is determined by the mold, therefore the first pitch D1 will not exist a difference on the combination. Thus the invention can ensure the accuracy of product assembly and atomization effect.

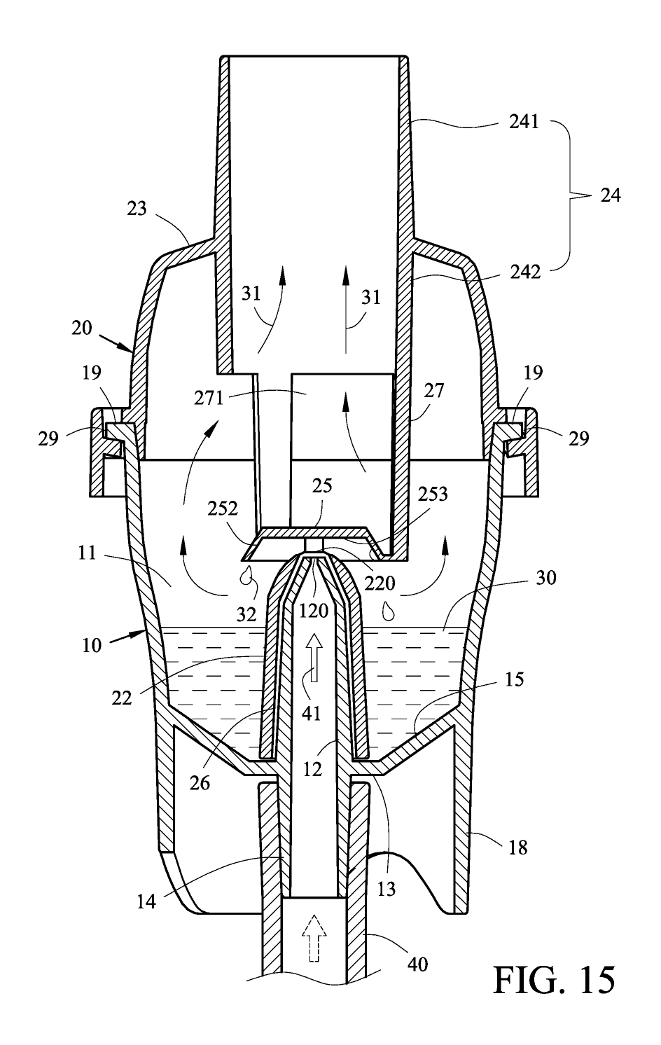

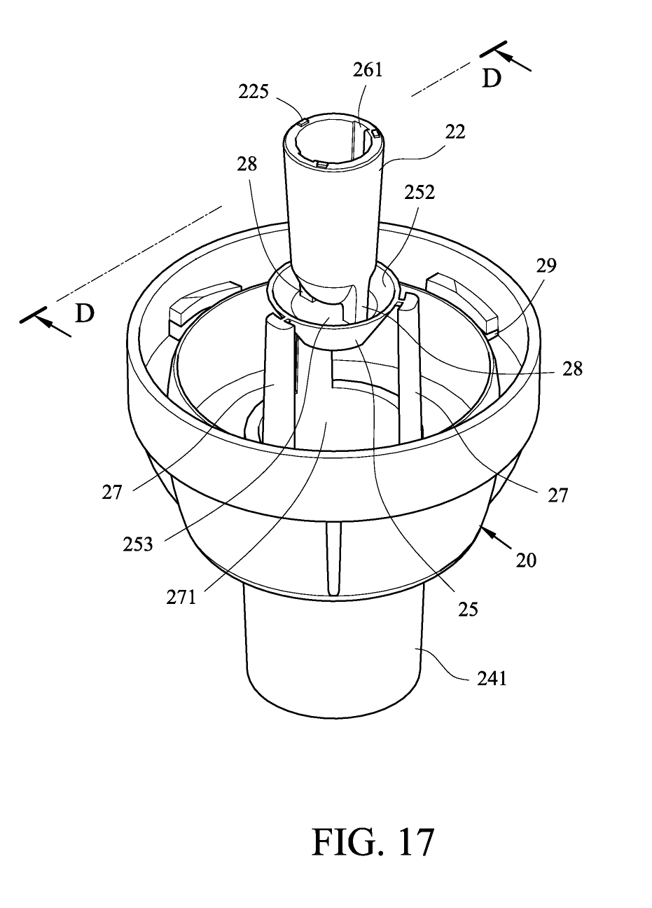

Examples of the connecting manner of the aerosol outlet tube 24 and the conical cover 25 will be illustrated below, the aerosol outlet tube 24 is connected to the conical cover 25 by a plurality of first connecting ribs 27, each pair of the adjacent connecting ribs 27 can form an aerosol window 271, the height of the conical cover 25 is in the range of 3 mm to 8 mm; wherein the aerosols 31 can enter the aerosol outlet tube 24 from the aerosol window 271, and the aerosols 31 can be outputted from the aerosol outlet tube 24 (as shown in FIG. 15 and FIG. 17).

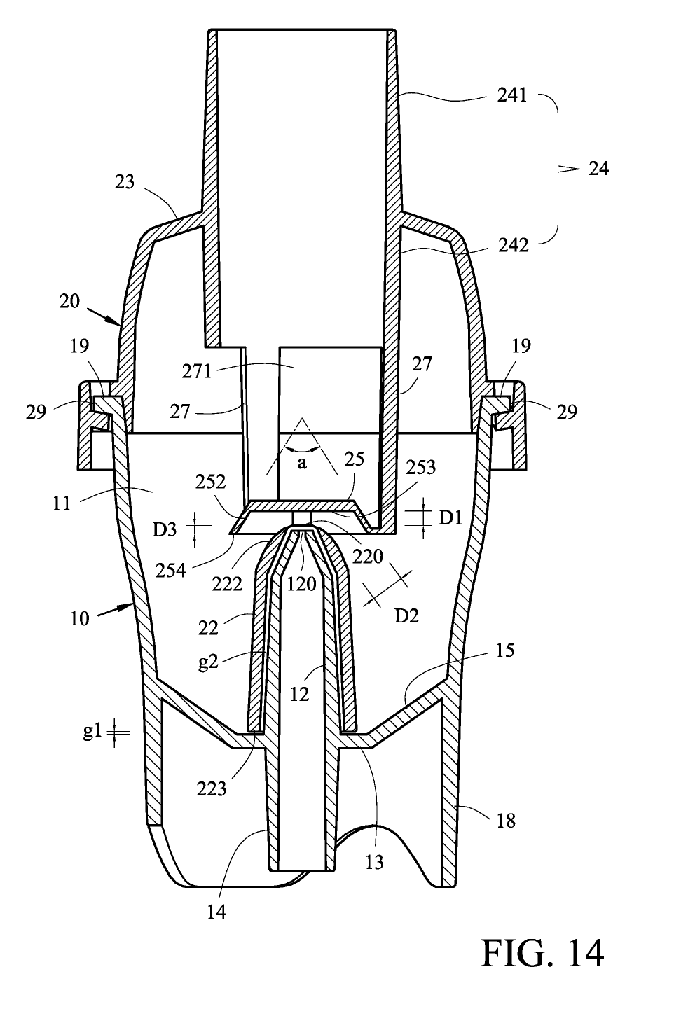

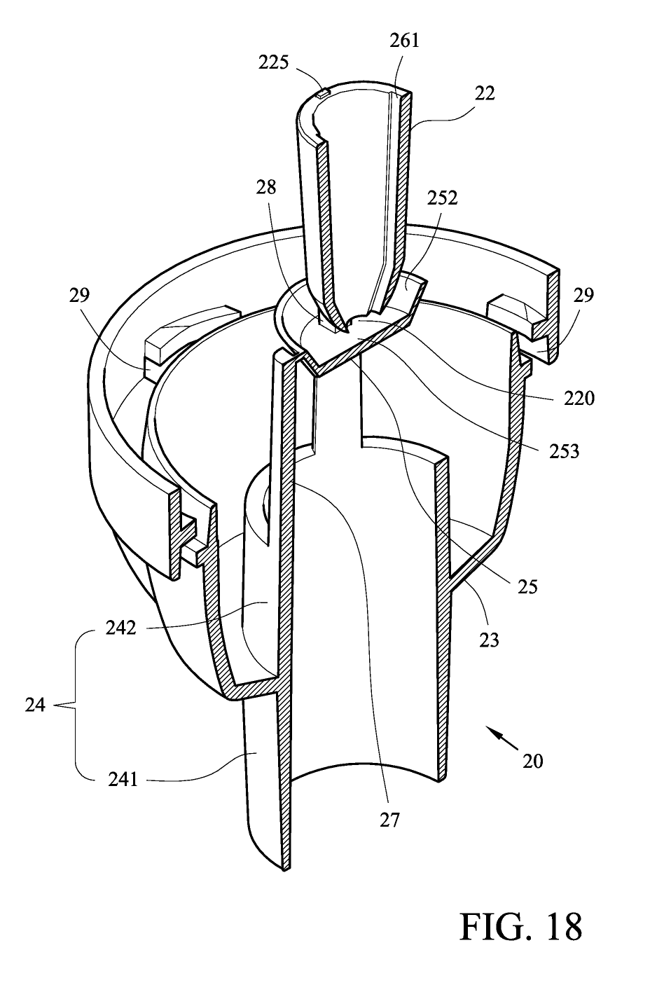

Examples of the connecting manner of the second nozzle 22 and the conical cover 25 will be illustrated below, the conical cover 25 is connected to the second nozzle 22 by a plurality of second connecting ribs 28 (as shown in FIG. 17 to FIG. 18). Moreover, the first pitch D1 formed between the impacting surface 253 and the second nozzle hole 220 is in the range of 1 mm to 3 mm, the second pitch D2 formed between the annular inclined surface 252 and the top end 222 of the second nozzle 22 is in the range of 1.5 mm to 5 mm, the impacting surface 253 is located on the inside of the conical cover 25, most of larger droplets 32 will be sprayed to the annular inclined surface 252 of the conical cover 25, and therefore can reduce the droplets 32 to attach to the inner wall of the medical nebulizer. Thus, the invention can further reduce the remaining amount of the liquid 30 (as shown in FIG. 14 to FIG. 15).

Examples of the positional relationship of the second nozzle hole 220 and the annular inclined surface 252 will be illustrated below, a third pitch D3 is formed between the second nozzle hole 220 and the bottom edge 254 of the annular inclined surface 252, the third pitch D3 is in the range of 0.1 mm to 3 mm, the annular inclined surface 252 of the conical cover 25 forms an angle a, the angle a is in the range of 40 degrees to 70 degrees; the conical cover 25 can form a suitable space of aerosol escaping by the annular inclined surface 252 and the second nozzle 22, the annular inclined surface 252 is helpful to the collection of larger droplets 32 and the dropping of larger droplets 32. Thus, the invention is optimized between the escaping amount of aerosols 31 and the collection amount of larger droplets 32 (as shown in FIG. 13 to FIG. 15).

Examples of a first practice manner of the fluid passage 26 will be illustrated below, a first gap g1 is formed between an end edge 223 of the second nozzle 22 and the bottom portion 13 of the first housing 10, the fluid passage 26 can be formed by a second gap g2 which located between the first nozzle 12 and the second nozzle 22 (as shown in FIG. 13 to FIG. 15).

Examples of a second practice manner of the fluid passage 26 will be illustrated below, a first gap g1 is formed between an end edge 223 of the second nozzle 22 and the bottom portion 13 of the first housing 10, the second nozzle 22 is provided with a plurality of inner grooves 261 to form the fluid passage 26 (as shown in FIG. 14, FIG. 15, FIG. 17 and FIG. 18).

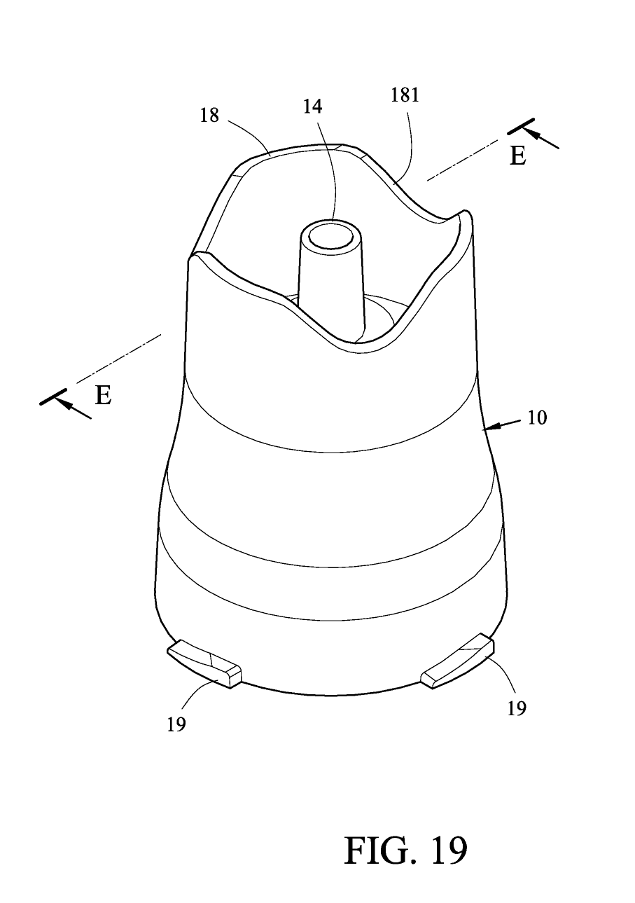

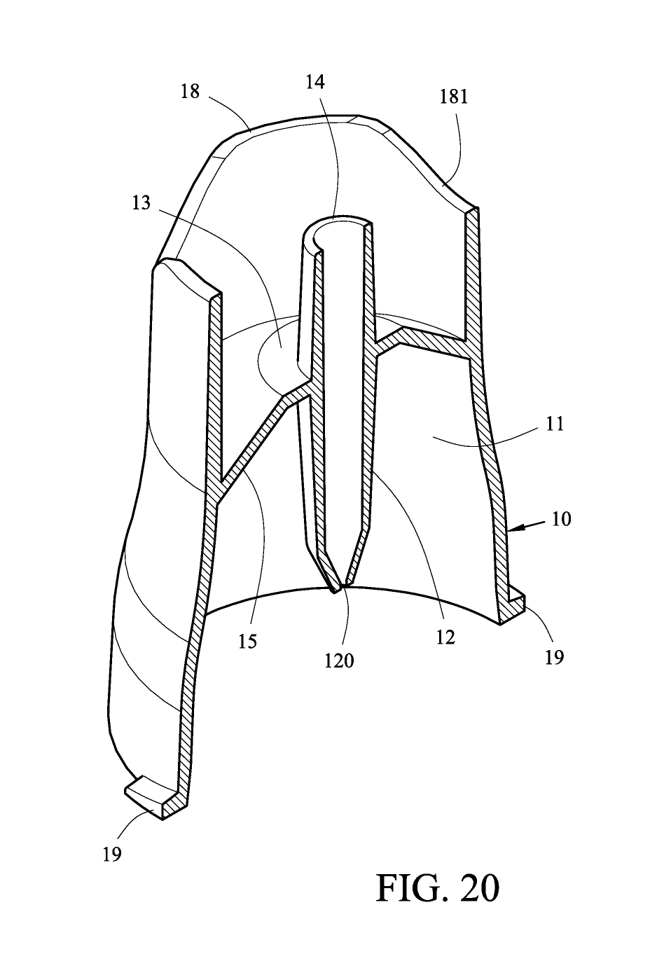

Examples of the structural change of the first housing 10 and the second housing 20 will be illustrated below, the bottom portion 13 of the first housing 10 has an inclined extension surface 15 to reduce the remaining amount of the liquid 30 (as shown in FIG. 19 to FIG. 20). Moreover, the end edge 223 of the second nozzle 22 has a plurality of blocks 225 to ensure a smallest distance of the first gap g1 (as shown in FIG. 14, FIG. 17 and FIG. 18). Furthermore, the first housing 10 further has an annular stand 18, the length of the annular stand 18 is larger than the length of the gas inlet tube 14, and the annular stand 18 has a plurality of notches 181 (as shown in FIG. 19 to and FIG. 20).

Examples of the structural change of the second housing 20 will be illustrated below, the aerosol outlet tube 24 has a first outlet tube 241 and a second outlet tube 242, the first outlet tube 241 is located on outside of the top portion 23 of the second housing 20, the second outlet tube 242 is located on inside of the top portion 23 of the second housing 20; when the medical nebulizer of the invention is tilted, the second outlet tube 242 can prevent the liquid 30 flow from the second housing 20 (as shown in FIG. 13 to and FIG. 18).

Examples of the combination manner of the first housing 10 and the second housing 20 will be illustrated below, the first housing 10 has a plurality of positioning blocks 19, the second housing 20 has a plurality of positioning slots 29; when the first housing 10 and the second housing 20 relatively rotate to each other, each of the positioning blocks 19 can enter into each of the positioning slots 29, or each of the positioning blocks 19 can escape from each of the positioning slots 29 (as shown in FIG. 13 to and FIG. 20).

What is claimed is:

1. A medical nebulizer with improved structure, the medical nebulizer comprising:
a first housing (10) having a first containing space (11), the first containing space (11) is provided with a first nozzle (12), the first nozzle (12) has a first nozzle hole (120), a bottom portion (13) of the first housing (10) outwardly extended a gas inlet tube (14), the gas inlet tube (14) is fluid communicated with the first nozzle (12);
a second housing (20), the second housing (20) is provided with a second nozzle (22), the second nozzle (22) has a second nozzle hole (220), the top portion (23) of the second housing (20) has a aerosol outlet tube (24), a conical cover (25) is connected with the aerosol outlet tube (24), the second nozzle (22) is connected with the conical cover (25), the inside of the conical cover (25) has a protrusion (251) and an annular inclined surface (252), the protrusion (251) formed a impacting surface (253) for producing aerosol;
wherein the second housing (20) is separable assembled to the first housing (10), the second nozzle (22) is located on the outside of the first nozzle (12), a fluid passage (26) is formed between the first nozzle (12) and the second nozzle (22), the first nozzle hole (120) is located on the inside of the second nozzle hole (220), the impacting surface (253) is located above the second nozzle hole (220), a first pitch (D1) is formed between the impacting surface (253) and the second nozzle hole (220), a second pitch (D2) is formed between the annular inclined surface (252) and the top end (222) of the second nozzle (22), the position of the second nozzle hole (220) is higher than the bottom edge (254) of the annular inclined surface (252).

2. The medical nebulizer with improved structure of claim 1, wherein the aerosol outlet tube (24) is connected to the conical cover (25) by a plurality of first connecting ribs (27), each pair of the adjacent connecting ribs (27) formed an aerosol window (271), the height of the conical cover (25) is in the range of 3 mm to 8 mm.

3. The medical nebulizer with improved structure of claim 1, wherein the conical cover (25) is connected to the second nozzle (22) by a plurality of second connecting ribs (28), the first pitch (D1) is in the range of 1 mm to 3 mm, the second pitch (D2) is in the range of 1.5 mm to 5 mm.

4. The medical nebulizer with improved structure of claim 1, wherein a third pitch (D3) is formed between the second nozzle hole (220) and the bottom edge (254) of the annular inclined surface (252), the third pitch (D3) is in the range of 0.1 mm to 3 mm.

5. The medical nebulizer with improved structure of claim 1, wherein the annular inclined surface (252) of the conical cover (25) can form an angle (a), the angle (a) is in the range of 40 degrees to 70 degrees.

6. The medical nebulizer with improved structure of claim 1, wherein a first gap (g1) is formed between an end edge (223) of the second nozzle (22) and the bottom portion (13) of the first housing (10), the fluid passage (26) can be formed by a second gap (g2) which located between the first nozzle (12) and the second nozzle (22).

7. The medical nebulizer with improved structure of claim 1, wherein a first gap (g1) is formed between an end edge (223) of the second nozzle (22) and the bottom portion (13) of the first housing (10), the second nozzle (22) is provided with a plurality of inner grooves (261) for forming the fluid passage (26).

8. The medical nebulizer with improved structure of claim 1, wherein the bottom portion (13) of the first housing (10) has an inclined extension surface (15), an end edge (223) of the second nozzle (22) has a plurality of blocks (225); the first housing (10) further has an annular stand (18), the length of the annular stand (18) is larger than the length of the gas inlet tube (14), and the annular stand (18) has a plurality of notches (181).

9. The medical nebulizer with improved structure of claim 1, wherein the aerosol outlet tube (24) has a first outlet tube (241) and a second outlet tube (242), the first outlet tube (241) is located on outside of the top portion (23) of the second housing (20), the second outlet tube (242) is located on inside of the top portion (23) of the second housing (20).

10. The medical nebulizer with improved structure of claim 1, wherein the first housing (10) has a plurality of positioning blocks (19), the second housing (20) has a plurality of positioning slots (29); when the first housing (10) and the second housing (20) relatively rotate to each other, each of the positioning blocks (19) can enter into each of the positioning slots (29), or each of the positioning blocks (19) can escape from each of the positioning slots (29).

11. A medical nebulizer with improved structure, the medical nebulizer comprising:
a first housing (10) having a first containing space (11), the first containing space (11) is provided with a first nozzle (12), the first nozzle (12) has a first nozzle hole (120), a bottom portion (13) of the first housing (10) outwardly extended a gas inlet tube (14), the gas inlet tube (14) is fluid communicated with the first nozzle (12);
a second housing (20), the second housing (20) is provided with a second nozzle (22), the second nozzle (22) has a second nozzle hole (220), the top portion (23) of the second housing (20) has a aerosol outlet tube (24), a conical cover (25) is connected with the aerosol outlet tube (24), the second nozzle (22) is connected with the conical cover (25), the inside of the conical cover (25) has an annular inclined surface (252) and a impacting surface (253) for producing aerosol;
wherein the second housing (20) is separable assembled to the first housing (10), the second nozzle (22) is located on the outside of the first nozzle (12), a fluid passage (26) is formed between the first nozzle (12) and the second nozzle (22), the first nozzle hole (120) is located on the inside of the second nozzle hole (220), the impacting surface (253) is located above the second nozzle hole (220), a first pitch (D1) is formed between the impacting surface (253) and the second nozzle hole (220), a second pitch (D2) is formed between the annular inclined surface (252) and the top end (222) of the second nozzle (22), the position of the second nozzle hole (220) is higher than the bottom edge (254) of the annular inclined surface (252).

12. The medical nebulizer with improved structure of claim 11, wherein the aerosol outlet tube (24) is connected to the conical cover (25) by a plurality of first connecting ribs (27), each pair of the adjacent connecting ribs (27) formed an aerosol window (271), the height of the conical cover (25) is in the range of 3 mm to 8 mm.

13. The medical nebulizer with improved structure of claim 11, wherein the conical cover (25) is connected to the second nozzle (22) by a plurality of second connecting ribs (28), the first pitch (D1) is in the range of 1 mm to 3 mm, the second pitch (D2) is in the range of 1.5 mm to 5 mm.

14. The medical nebulizer with improved structure of claim 11, wherein a third pitch (D3) is formed between the second nozzle hole (220) and the bottom edge (254) of the annular inclined surface (252), the third pitch (D3) is in the range of 0.1 mm to 3 mm.

15. The medical nebulizer with improved structure of claim 11, wherein the annular inclined surface (252) of the conical cover (25) can form an angle (a), the angle (a) is in the range of 40 degrees to 70 degrees.

16. The medical nebulizer with improved structure of claim 11, wherein a first gap (g1) is formed between an end edge (223) of the second nozzle (22) and the bottom portion (13) of the first housing (10), the fluid passage (26) can be formed by a second gap (g2) which located between the first nozzle (12) and the second nozzle (22).

17. The medical nebulizer with improved structure of claim 11, wherein a first gap (g1) is formed between an end edge (223) of the second nozzle (22) and the bottom portion (13) of the first housing (10), the second nozzle (22) is provided with a plurality of inner grooves (261) for forming the fluid passage (26).

18. The medical nebulizer with improved structure of claim 11, wherein the bottom portion (13) of the first housing (10) has an inclined extension surface (15), an end edge (223) of the second nozzle (22) has a plurality of blocks (225); the first housing (10) further has an annular stand (18), the length of the annular stand (18) is larger than the length of the gas inlet tube (14), and the annular stand (18) has a plurality of notches (181).

19. The medical nebulizer with improved structure of claim 11, wherein the aerosol outlet tube (24) has a first outlet tube (241) and a second outlet tube (242), the first outlet tube (241) is located on outside of the top portion (23) of the second housing (20), the second outlet tube (242) is located on inside of the top portion (23) of the second housing (20).

20. The medical nebulizer with improved structure of claim 11, wherein the first housing (10) has a plurality of positioning blocks (19), the second housing (20) has a plurality of positioning slots (29); when the first housing (10) and the second housing (20) relatively rotate to each other, each of the positioning blocks (19) can enter into each of the positioning slots (29), or each of the positioning blocks (19) can escape from each of the positioning slots (29).
